(19) United States
(12) Patent Application Publication
(10) Pub. No.: US 2025/0275862 A1
(43) Pub. Date: Sep. 4, 2025

(54) Lip-based user interface system

(71) Applicant: THE UNIVERSITY OF CHICAGO, Chicago, IL (US)

(72) Inventors: Pedro Lopes, Chicago, IL (US); Arata Jingu, Chicago, IL (US); Yudai Tanaka, Chicago, IL (US)

(21) Appl. No.: 19/060,948

(22) Filed: Feb. 24, 2025

Related U.S. Application Data
(60) Provisional application No. 63/561,168, filed on Mar. 4, 2024.

(51) Int. Cl. A61F 4/00; G06F 3/01

(52) U.S. Cl. CPC A61F 4/00; G06F 3/011; G06F 3/016; G06F 3/017

(57) ABSTRACT

Systems and methods are provided to allow a user's lip to be used as a high temporal spatial resolution input and output surface. A stimulator is applied to the lip that includes a plurality of electrodes. These electrodes (e.g., a first subset) can be used to provide high-resolution electrohaptic stimulation to the lip, allowing the lip to be used to provide outputs of a user interface. These electrodes (e.g., a second subset) can also be used via capacitive or other sensing methods to detect the location of contact of the user's lip by the user's tongue and/or opposite lip, allowing the lip to be used to provide inputs to a user interface. Such a lip-based user interface provides hands-and eyes-free bidirectional information flow between a user and electronic systems, and does so in a manner that minimally impedes speech or eating.

### CROSS-REFERENCE TO RELATED APPLICATION

This application claims priority to U.S. Provisional Patent Application No. 63/561,168, filed on Mar. 4, 2024, the contents of which are hereby incorporated by reference in their entirety.

### STATEMENT REGARDING FEDERALLY SPONSORED RESEARCH OR DEVELOPMENT

This invention was made with government support under 2047189 awarded by the National Science Foundation. The government has certain rights in the invention.

### BACKGROUND

It is desirable to provide a human-computer interface (HCI) that is able to provide outputs to and receive inputs from users while being minimally intrusive, so that a user can engage with their environment normally (e.g., using their hands) while also using the HCI and without the presence of the HCI interfering with their interaction with the environment. It is also desirable to provide HCIs that allow individuals with diminished physical capabilities (e.g., individuals without the use of their legs and/or arms) to interact with systems (e.g., motorized wheelchairs or other assistive systems) without overly monopolizing their physical capabilities (e.g., allowing an individual to control a motorized wheelchair while simultaneously using their arms to manipulate objects in their environment).

Sound is the most common modality used to realize both eyes and hands-free HCIs. Sound-based interactions that feature both input and output have become common in smartphones and smart speakers, which provide micro-interactions for setting timers, playing music, etc. However, such an interface is not usable in all contexts. For example, a sound-based HCI interferes with external sounds and is harder to use in public settings or in a multi-party conversation.

Alternatively, hands- or eyes-free HCIs have been implemented using some part of the user's body as the medium of interaction for the HCI, e.g., the user can both feel output and can respond with input through a limb. For instance, this can be accomplished by actuating the user's fingers via an actuated smartphone screen; users responded via gestures on the same screen, resulting in symmetric I/O. In another example, users with visual impairments were enabled to play interactive games by delivering tactile output on the palm (via a shape display) while providing touch input on the back of the hand. These devices allow users not only to interact eyes-free but even with a high bandwidth (e.g., Gesture Output offered a full A-Z alphabet). However, these approaches also require the user to hold on to a device. Proprioceptive Interaction leveraged muscle sensing and electrical muscle stimulation to realize information input and output via the same limb. In yet further examples, simultaneous muscle stimulation and sensing was leveraged to create notifications or share information across two users. While these systems realize eyes-free I/O without the need for sounds, they all require the user's hands to operate, which makes these approaches not hands-free.

### SUMMARY

In a first aspect, a system is provided that includes a stimulator, the stimulator including: (i) a flexible substrate having a first surface and a second surface opposite the first surface; (ii) a first set of electrodes disposed on the first surface of the flexible substrate; (iii) a ground electrode disposed on the first surface of the flexible substrate; and (iv) a second set of electrodes disposed on the second surface of the flexible substrate, wherein the stimulator is mountable to a lip, wherein the first set of electrodes and ground electrode are operable to provide electrohaptic stimulation to the lip when the stimulator is mounted thereto, and wherein the second set of electrodes are operable to detect a location of contact between the lip and at least one of an opposite lip or a tongue when the stimulator is mounted to the lip.

In a second aspect, a method is provided that includes: (i) applying a stimulator to a lip of a wearer; (ii) using a first set of electrodes and a ground electrode of the stimulator, delivering a first electrohaptic stimulus to the lip; and (iii) using a second set of electrodes of the stimulator, detecting a location of contact between the lip and at least one of an opposite lip or a tongue of the wearer.

In a third aspect, a method is provided that includes: (i) applying a flexible stimulator to a lip of a wearer; and (ii) using a set of electrodes and a ground electrode of the flexible stimulator, delivering a first electrohaptic stimulus to the lip.

In a fourth aspect, a non-transitory computer readable medium is provided having stored thereon program instructions executable by at least one processor to cause the at least one processor to perform the method of the second or third aspects.

In a fifth aspect, system is provided that includes: (i) a controller comprising one or more processor, and (ii) a non-transitory computer readable medium having stored thereon program instructions executable by the controller to cause the controller to perform the method of the second or third aspects.

### BRIEF DESCRIPTION OF THE DRAWINGS

The accompanying drawings are included to provide a further understanding of the system and methods of the disclosure and are incorporated in and constitute a part of this specification. The drawings illustrate one or more embodiment(s) of the disclosure, and together with the description serve to explain the principles and operation of the disclosure.

FIG. 1 depicts aspects of a system for providing outputs to a user and receiving inputs from the user via the user's lip, in accordance with example embodiments.

FIG. 2 depicts aspects of the construction and use of a lip-based user interface system, in accordance with example embodiments.

FIG. 3 depicts aspects of an example system.

FIG. 4 depicts aspects of an example method.

FIG. 5 depicts aspects of an example method.

FIG. 6 depicts aspects of an experiment.

FIG. 7 depicts aspects of an experiment.

FIG. 8 depicts aspects of an experiment.

FIG. 9 depicts aspects of an experiment.

FIG. 10 depicts aspects of an experiment.

FIG. 11 depicts aspects of an experiment.

FIG. 12 depicts aspects of an experiment.

FIG. 13 depicts experimental results.

FIG. 14 depicts aspects of an experiment.

FIG. 15 depicts experimental results.

FIG. 16 depicts aspects of experimental applications.

### DETAILED DESCRIPTION

The following detailed description describes various features and functions of the disclosed embodiments with reference to the accompanying figures. The illustrative embodiments described herein are not meant to be limiting. It may be readily understood that certain aspects of the disclosed embodiments can be arranged and combined in a wide variety of different configurations, all of which are contemplated herein.

### I. Overview

It is desirable in a variety of applications to provide a bidirectional human-computer interface (HCI) that allows a user to provide input information (e.g., commands) to a computer system, and to receive output information from the computer system, while leaving the user's hands, legs, eyes, and other senses/body parts free to interact with the user's environment unimpeded. Such a non-intrusive HCI can also be beneficial for use by users with diminished physical capabilities (e.g., due to amputation, spinal cord injury, etc.) who might not be able to use their arms, legs, etc. to operate conventional HCIs. For such users, it can be particularly important to minimize intrusion on the user's existing physical capabilities, as they may have fewer options to perform activities of daily living while also operating an HCI (e.g., a person who is unable to use their legs may need to use their arms to hold an object while also operating an HCI to control a motorized wheelchair). For users with diminished physical capabilities, it can also be beneficial for an HCI to operate using the muscles and skin of the face, eyes, or other aspects of the head or neck, as the ability to use and sense via these portions of the body are often retained even by individuals with extreme levels of impairment (e.g., high level spinal cord injury, advanced cases of amyotrophic lateral sclerosis).

The present disclosure provides embodiments of such an unobtrusive HCI, which includes placing a flexible array of electrodes on one or both lips of a user. These electrodes can be operated to deliver electrohaptic stimulation to the lips, providing output information to the user, while also detecting the location of contact between the lip and the tongue or opposite lip of the user, receiving input information from the user. The lip has high tactile acuity, allowing the embodiments described herein to be used to provide high-bandwidth output information to a user. Additionally, the lips and tongue are capable of very fine motion, allowing the embodiments described herein to be used to receive high-bandwidth input information from a user. Additionally, the lips and tongue are able to be controlled and sensed even by individuals with very high-level spinal cord injury, advanced ALS, or other advanced conditions that affect the ability to sense and to use parts of the body. Regardless of the user's degree of use of their body, the HCI embodiments described herein provide a high-bandwidth bidirectional information channel that is minimally encumbering of a user's senses and ability to interact with their environment, allowing them to interact with an electronic system while still seeing and hearing their environment normally and using their arms and legs to interact with that environment in an unencumbered manner.

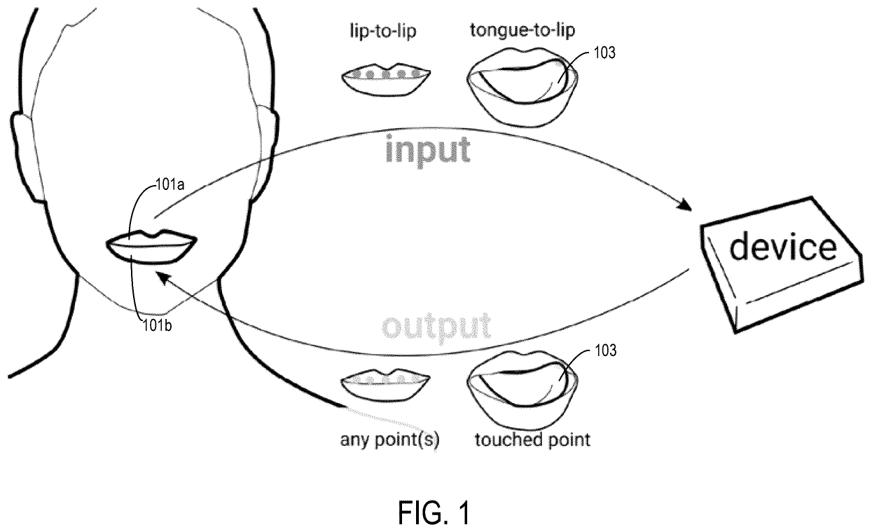

FIG. 1 depicts aspects of the operation of such a system. Inputs can be detected by detecting the location of contact between the system, disposed on the user's lip 101a, and the user's tongue 103 or opposite lip 101b. Such a detected location input can include the identity of one of the discrete electrodes of the system that is most closely being touched, the identity of a set of the discrete electrodes of the system that are being simultaneously touched, the location and optionally extent of contact of a tongue or opposite lip with the system (e.g., relative an internal location metric), and optionally a detected identity of the body part touching the system (tongue vs. opposite lip). Outputs can be provided by operating one or more of the electrodes of the system to provide electrohaptic stimulation to respective portions of the user's lip.

The locations of the electrodes used to detect the location of contact and the electrodes used to provide electrohaptic stimuli could correspond to each other (e.g., the number of the sensing electrodes and the stimulation electrodes could be the same, and corresponding electrodes could fully or partially overlap). In such examples, some of the output information provided via the stimulation electrodes could take the form of feedback, indicating that the corresponding sensing electrode(s) have detected a touch from the user's tongue/opposite lip, that the corresponding sensing electrode(s) have been contacted long or firmly enough to register as an input (e.g., a ‘short button press,’ a ‘long button press’), or to provide some other electrohaptic feedback to guide the user's interaction with the HCI.

Such a system can be implemented as a flexible stimulator that is mountable to a user's lip and that includes (i) electrodes that are operable, when the stimulator is mounted to the user's lip, to detect the location of contact of the user's tongue or opposite lip with the stimulator (e.g., via capacitive sensing) and (ii) electrodes that are operable, when the stimulator is mounted to the user's lip, to deliver electrohaptic stimuli to the user's lip. The sensing and stimulation electrodes may be different sets of electrodes, or may be operated in a multiplexed fashion such that a single electrode may, at different points in time, be used to provide electrohaptic stimulus and to detect contact with the user's tongue/opposite lip.

Where the electrodes are two separate sets of sensing and stimulation electrodes, the two sets of electrodes may be insulated from each other by being disposed on opposite sides of a flexible substrate (e.g., a sheet of flexible polymer material). This way, the effect of the underlying lip on the capacitive sensing of contact with the tongue/opposite lip can be reduced, and the effect of electrohaptic stimulation of the underlying lip on portions of the tongue/opposite lip that are in contact with the flexible stimulator can be reduced. Further, such a double-sided construction can facilitate quick, consistent, low-cost fabrication of the flexible stimulator.

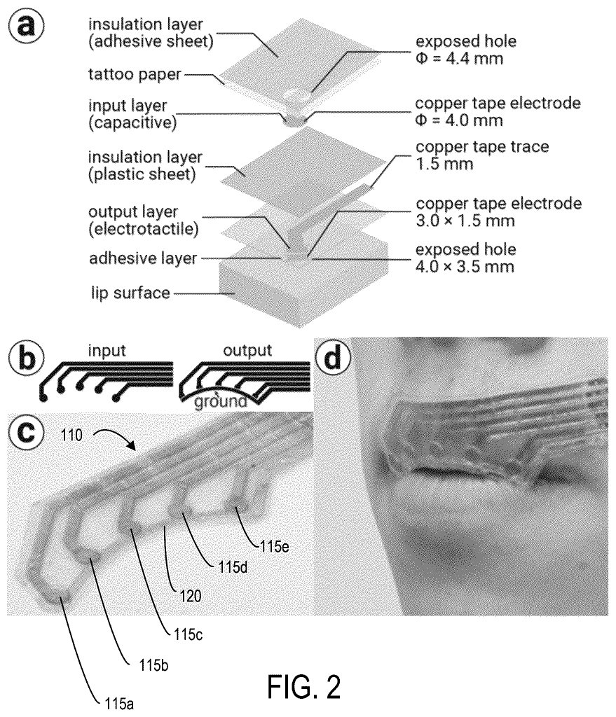

FIG. 2 depicts aspects of an example of such a flexible stimulator 110, which includes five stimulation electrodes and an arcuate ground electrode 120 in common between the stimulation electrodes (“output layer”) and five sensing electrodes 115a-e (“input layer,” these electrodes occlude view of the stimulation electrodes in FIG. 2 (c)) that correspond to the stimulation electrodes and overlap therewith.

Note that the specific embodiment depicted in FIG. 2 is intended as a non-limiting example embodiment of the present disclosure, provided for ease of illustration of the various aspects of the present disclosure. Alternative aspects or configurations of a device or system as described herein (e.g., having different numbers of electrodes or layers or material, having differently-dimensioned electrodes or other components, having different material compositions) are possible and encompassed within the scope of the present disclosure.

For example, the sensor electrodes and stimulation electrodes of a flexible stimulator as described herein could be formed on respective different flexible substrate materials (e.g., by adhering metal foils thereto, by printing or otherwise depositing conductive materials thereon, etching or otherwise removing conductive materials therefrom). These separate electrode-and-flexible substrate assemblies could then be adhered or otherwise coupled together to form a flexible stimulator as described herein. In such an example embodiment, both sets of electrodes thereof can be considered to be ‘disposed on’ respective opposite surfaces of a single flexible substrate (that ‘single flexible substrate’ being the combination of the two original flexible substrates in examples wherein the two original flexible substrates were coupled together with no electrodes there between). Alternatively, a flexible insulating layer could be disposed between the two electrode-and-flexible substrate assemblies when coupling them together (e.g., the layer of insulating material could be or have disposed thereon an adhesive to facilitate such coupling).

Indeed, a flexible stimulator as described herein could lack a central flexible insulating layer disposed between separate sets of sensor and stimulation electrodes. For example, a single set of electrodes could be used, in a time-multiplexed manner, to both sense the location of contact with a tongue lip and to deliver electrohaptic stimulation to a lip on which such electrodes are disposed. In yet another example, the two sets of electrodes could be disposed on the same side of the same supportive flexible substrate, and their respective operations as sensor and stimulator achieved by some other means (e.g., by providing a layer of insulation over the sensor electrodes but not the stimulation electrodes, allowing the stimulation electrodes to more easily deliver electrohaptic stimulation to an underlying lip while reducing interference from such an underlying lip in the use of the sensor electrodes to detect the location of contact with an overlying tongue or opposite lip). In some examples, a flexible stimulator as described herein could lack sensor electrodes entirely, and be used only to provide electrohaptic output to a user, via stimulation of the user's lip, without also being operable or operated to detect the location of contact of that lip with the user's tongue or opposite lip. In some examples, a system as described herein could include two flexible stimulators, or a single flexible stimulator, that is configured to provide electrohaptic stimuli to both lips of a wearer and/or to detect contact with the tongue or opposite lip for both lips of the wearer.

In some examples, the electrodes of a flexible stimulator as described here could be implemented as discrete wires or other conductive materials (e.g., indium tin oxide or other transparent conductive materials), rather than as metal foils or other conductive materials that also form the electrodes of the stimulator. Indeed, a flexible stimulator as described herein could include, disposed on the flexible substrate thereof, electronic switches (e.g., thin film transistors, discrete surface-mount transistors), passive components (e.g., capacitors, resistors, which may be formed from metal or other material traces disposed on layer(s) of the substrate or may be discrete components disposed thereon), microcontrollers, or other electronics configured to operate the electrodes to deliver stimuli, to detect the location of contact with a tongue/lip, or to perform other operations as described herein.

In some examples, a flexible stimulator as described herein could include multiple grounds. For example, such a system could include a respective ground for each stimulator electrode thereof, rather than the single common ground illustrated in FIG. 2. Such a system could additionally or alternatively include ground or other types of electrodes for use with the sensor electrodes to detect the location of contact of a tongue or opposite lip, e.g., used as return electrodes in order to detect such location of contact resistively, galvanically, or in some other multi-electrode manner.

A flexible stimulator as described herein could include more or fewer sensor and/or stimulation electrodes than the five depicted in FIG. 2. The number of electrodes could be selected based on a variety of factors, including system cost, the ability of users to contact accurately individual electrode locations with their tongue/opposite lip for a given electrode size/spacing, the ability of users to distinguish accurately electrohaptic stimuli from different electrodes for a given electrode size/spacing, or other considerations. For example, where the user interface implemented using such a system includes the user contacting individual electrodes, and distinguishing between stimuli delivered from individual electrodes, the system could include between four and nine sensor electrodes and/or stimulation electrodes, inclusive.

A flexible stimulator as described herein could include the same number of sensor electrodes as stimulation electrodes. Where the number of both types of electrodes is the same, the sets of electrodes could correspond to each other such that each sensor electrode fully or partially overlaps with a corresponding one of the stimulation electrodes (e.g., as depicted in FIG. 2). Where the sensor and stimulation electrodes overlap in this manner, electrohaptic stimulation provided by a stimulation electrode can be used as feedback to guide a user in correctly contacting the corresponding sensor electrode and/or in interacting with the corresponding sensor electrode in the correct manner (e.g., by providing electrohaptic feedback when the user initially contacts and/or remains in contact with the corresponding sensor electrode, by providing electrohaptic feedback when the user contacts the corresponding sensor electrode for a sufficient amount of time to register as a “button press” gesture or other gesture).

Electrohaptic stimulation can be provided via a particular stimulation electrode by applying a voltage between the stimulation electrode and a ground electrode, e.g., between 10 and 20 volts. Applying the stimulation can include injecting a specified current or time-varying current waveform, e.g., a square wave with respect to current, using a controlled-current source. The magnitude of the applied electrohaptic stimulation can be adjusted by adjusting the amplitude of the injected current, e.g., between 1 mA and 10 mA. In practice, an injected current amplitude of less than 5 milliamps is sufficient to provoke the perception of a haptic stimulus in the lip without creating discomfort. The amplitude of the injected current (e.g., default level of current, a maximum level of current) can be set by applying a range of levels of stimulation to a user, and allowing the user to select which level is preferred (e.g., that evokes a haptic perception without discomfort. A particular stimulation electrode could be operated to provide only one level of electrohaptic stimulation by injecting current there through at only a single amplitude. Alternatively, multiple different levels of electrohaptic stimulation may be provided by injecting current there through at corresponding different amplitudes.

Electrohaptic stimulation could be provided through single stimulation electrodes at a time or through multiple. Providing simultaneous electrohaptic stimulation through multiple stimulation electrodes could include injecting current through the multiple stimulation electrodes at the same time or time-multiplexing the current injected there through in a manner that a user perceives as being simultaneous. The spatial (across electrodes) and temporal pattern of provided electrohaptic stimulus can be specified according to a specified user interface to communication information to a wearer. This information can include information about the operation or contents of other systems (e.g., alerts from a user's phone, navigation instructions from a GPS system, instructions to tune an instrument). Additionally or alternatively, the information provided electrohaptically to a user can be related to the user's operation of the system, e.g., feedback information to guide a user in contacting the appropriate locations (e.g., sensor electrodes) on a flexible stimulator and/or to guide a user in performing desires control gestures on the flexible stimulator.

A set of sensor electrodes can be operated in a variety of ways to detect the location of contact of a user's tongue or opposite lip with the flexible stimulator. This can include using the sensor electrodes to detect contact with the tongue or opposite lip capacitively, resistively, or in some other manner. The sensor electrodes could also be used to detect whether a tongue or an opposite lip is contacting the flexible stimulator. Detecting the “location of contact” or a tongue or an opposite lip can include determining the identity of one of the discrete electrodes of the system that is most closely being touched, the identity of a set of the discrete electrodes of the system that are being simultaneously touched, the location and optionally extent of contact of a tongue or opposite lip with the system (e.g., relative an internal location metric). Detecting a single location of contact can include determining the identity of the electrode of the set of sensor electrodes that is in closest contact with the tongue/opposite lip (e.g., with respect to detected change in capacitance and/or resistance of the sensor electrodes) or determining a centroid, along the set of sensor electrodes, of the degree of contact of each of the sensor electrodes with the tongue/opposite lip (e.g., with respect to detected change in capacitance and/or resistance of the sensor electrodes).

A user interface for a variety of systems (e.g., a cellphone, a motorized wheelchair or other assistive system or device, a laptop or other computer, a home automation system, a DJ system, an instrument tuner) could be implemented in whole or in part using a flexible stimulator as described herein. This can include using a variety of ‘primitives’ to present information to/receive commands or other information from a user. For example, certain pre-specified patterns of electro-haptic stimulation could be provided to indicate, to a user, that corresponding events have occurred or to indicate some other information (e.g., stimulation of a specified pattern of stimulation electrodes could indicate that a doorbell is being rung; a pattern over time of stimulation moving along stimulation electrodes from left to right could indicate that a door has been unlocked while the pattern from the right to left could indicate that the door has been locked). Additionally or alternatively, certain patterns of contact between the flexible stimulator and a user's tongue or opposite lip could be detected (based on the detected location of contact over time) and interpreted as commands or other information input to a system (such detected, pre-specified patterns of contact may be referred to as “gestures”).

A system that includes the flexible stimulator (e.g., a motorized wheelchair or other assistive device) could implement such a user interface locally, generating the output information indicated to the user via the flexible stimulator and using the input commands and other information received from the user to operate the system (e.g., to control movement of the motorized wheelchair). Additionally or alternatively, a flexible stimulator-including system as describe herein could be in communication with one or more remote systems (e.g., cellphones, home automation systems, assistive system via a wired or wireless communications link) and could act as a user interface “peripheral” for such remote systems.

In such examples, the flexible stimulator system could act merely as a ‘pass-through:’ receiving indications of the spatial and temporal pattern of electrohaptic stimulation to provide to a user and implementing them, and detecting the location of contact of a user's tongue and/or opposite lip with the flexible stimulator and transmitting a representation of that detected location over time. Additionally or alternatively, the flexible stimulator-including system could communicate with the remote system in terms of more abstract information (e.g., door lock/unlock commands or status updates, volume change commands, tuner tone feedback outputs) and could map such information to patterns of electrohaptic stimulation/from gestures of contact with the stimulator performed by a user according to a pre-specified mapping pattern.

Output “gestures” can include a variety of spatial and/or temporal patterns of delivered electrohaptic stimulus. For example, providing electrohaptic stimulus via a pre-specified set of stimulation electrodes could indicate a corresponding idea or other information (e.g., a numerical value, the identity of a door or other object in a list of such objects). The pattern of stimulus could vary over time to indicate additional information. E.g., providing a ‘moving’ stimulus on each of the stimulation electrodes from left to right (or right to left) could indicate some information, with the direction of the ‘motion’ providing part of the information (e.g., motion to the left indicating that a lock is being unlocked, while motion to the right indicates that the lock is being locked). The pattern of provided stimulus could provide context for subsequently provided stimulus. For example, during a first period of time, a first pattern of stimulus could be provided to indicate that subsequent stimulus represents information about a specified system or device (e.g., about a doorbell, about a door lock), and then a second pattern of stimulus provided during a second, subsequent period of time to indicate the information about the system or device specified by the pattern of stimulus provided during the first period of time. Other patterns of output electrohaptic stimulus could be provided to indicate other information.

Input “gestures” can include a variety of spatial and/or temporal patterns of contact between a flexible stimulator mounted to a user's lip and the user's tongue or opposite lip. For example, input gestures can include contacting a specified location of the flexible stimulator (e.g., a specified one or more sensor electrode thereof), optionally by a specified one of the user's tongue or opposite lip and/or for more than a specified period of time. Input gestures can include motion, e.g., moving the tip of the user's tongue in contact with the flexible stimulator from one side to the other in a specified direction, or moving from one side to the other and then back in a single motion. Other patterns of contact with the flexible stimulator could be performed by a user (and subsequently detected by the flexible stimulator system) to detect other commands or information from the user.

### II. Example Systems

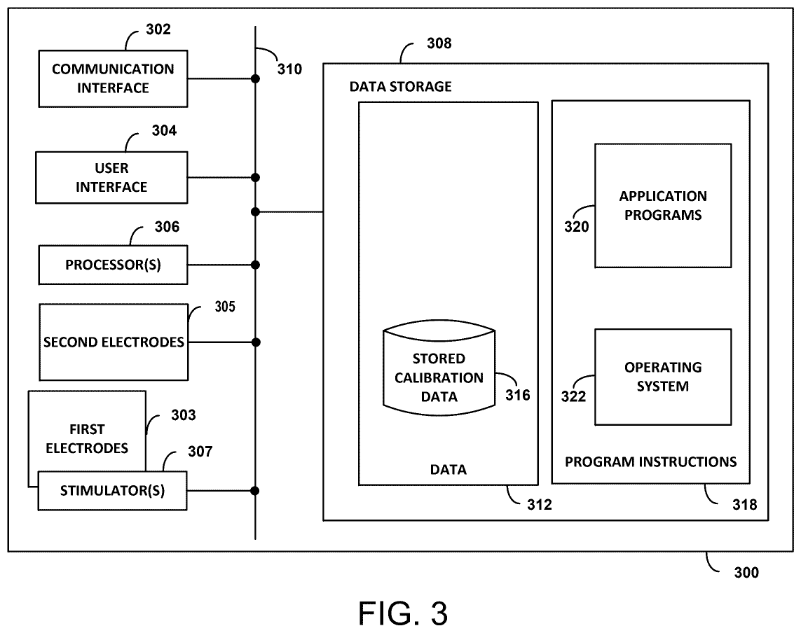

FIG. 3 illustrates an example system 300 that may be used to implement the methods and/or apparatus described herein. By way of example and without limitation, system 300 may be or include a computer (such as a desktop, notebook, tablet, or handheld computer, a server), elements of an wearable system (e.g., a system configured to be worn on one or both lips of a wearer), elements of an assistive device, or some other type of device or system or combination of devices and/or systems. It should be understood that elements of system 300 may represent a physical instrument and/or computing device such as a server, a particular physical hardware platform on which applications operate in software, or other combinations of hardware and software that are configured to carry out functions as described herein.

As shown in FIG. 3, system 300 may include a communication interface 302, a first set of electrodes 303 operable to deliver electrohaptic stimulation to a lip (e.g., stimulation electrodes configured to be applied to skin of a lip, ground electrode(s) configured to be applied to skin of the lip to provide a return path for stimulation currents injected via stimulation electrodes), a second set of electrodes 305 operable to detect the location of contact between a lip on which the electrodes are mounted and a tongue or an opposite lip (e.g., capacitive sensor electrodes), a user interface 304, one or more processors 306, one or more stimulators 307, and data storage 308, all of which may be communicatively linked together by a system bus, network, or other connection mechanism 310.

Communication interface 302 may function to allow system 300 to communicate, using analog or digital modulation of electric, magnetic, electromagnetic, optical, or other signals, with other devices (e.g., with systems providing sets of outputs to be delivered to a lip of a wearer of the system 300 and/or receiving sets of inputs generated by the wearer contacting their lip with their tongue and/or opposite lip), access networks, and/or transport networks. Thus, communication interface 302 may facilitate circuit-switched and/or packet-switched communication, such as plain old telephone service (POTS) communication and/or Internet protocol (IP) or other packetized communication. For instance, communication interface 302 may include a chipset and antenna arranged for wireless communication with a radio access network or an access point. Also, communication interface 302 may take the form of or include a wireline interface, such as an Ethernet, Universal Serial Bus (USB), or High-Definition Multimedia Interface (HDMI) port. Communication interface 302 may also take the form of or include a wireless interface, such as a WiFi, BLUETOOTH®, global positioning system (GPS), or wide-area wireless interface (e.g., WiMAX, 3GPP Long-Term Evolution (LTE), or 3GPP 5G). However, other forms of physical layer interfaces and other types of standard or proprietary communication protocols may be used over communication interface 302. Furthermore, communication interface 302 may comprise multiple physical communication interfaces (e.g., a WiFi interface, a BLUETOOTH® interface, and a wide-area wireless interface).

User interface 304 may function to allow system 300 to interact with a user, for example to receive input from and/or to provide output to the user. Thus, user interface 304 may include input components such as a keypad, keyboard, touch-sensitive or presence-sensitive panel, computer mouse, trackball, joystick, microphone, and so on. User interface 304 may also include one or more output components such as a display screen which, for example, may be combined with a presence-sensitive panel. The display screen may be based on CRT, LCD, and/or LED technologies, or other technologies now known or later developed. User interface 304 may also be configured to generate audible output(s), via a speaker, speaker jack, audio output port, audio output device, earphones, and/or other similar devices. The user interface 304 may be operable to permit a user to initiate a calibration procedure and to provide feedback related thereto (e.g., to indicate whether stimulation provided by the system provoked a haptic perception on the lip, to indicate that the lip is being contacted by a tongue or opposite lip), allowing stimulation magnitude calibration data to be generated and/or input, or to perform some other operation.

Processor(s) 306 may comprise one or more general purpose processors—e.g., microprocessors—and/or one or more special purpose processors—e.g., digital signal processors (DSPs), graphics processing units (GPUs), floating point units (FPUs), network processors, tensor processing units (TPUs), or application-specific integrated circuits (ASICs). Data storage 308 may include one or more volatile and/or non-volatile storage components, such as magnetic, optical, flash, or organic storage, and may be integrated in whole or in part with processor(s) 306 and/or with some other element of the system. Data storage 308 may include removable and/or non-removable components.

Processor(s) 306 may be capable of executing program instructions 318 (e.g., compiled or non-compiled program logic and/or machine code) stored in data storage 308 to carry out the various functions described herein. Therefore, data storage 308 may include a non-transitory computer-readable medium, having stored thereon program instructions that, upon execution by system 300, cause system 300 to carry out any of the methods, processes, or functions disclosed in this specification and/or the accompanying drawings. The execution of program instructions 318 by processor(s) 306 may result in processor 306 using data 312.

By way of example, program instructions 318 may include an operating system 322 (e.g., an operating system kernel, device driver(s), and/or other modules) and one or more application programs 320 (e.g., functions for executing the methods described herein) installed on system 300. Data 312 may include stored calibration data 316 (e.g., stored sets stimulation thresholds to induce haptic perceptions on the lip for each electrode of the array 303) that can be used to determine how to operate the simulator(s) 307 and/or first electrode(s) 303 to provide electro-haptic sensory stimulus to a user.

Application programs 320 may communicate with operating system 322 through one or more application programming interfaces (APIs). These APIs may facilitate, for instance, application programs 320 transmitting or receiving information via communication interface 302, receiving and/or displaying information on user interface 304, and so on.

Application programs 320 may take the form of “apps” that could be downloadable to system 300 through one or more online application stores or application markets (via, e.g., the communication interface 302). However, application programs can also be installed on system 300 in other ways, such as via a web browser or through a physical interface (e.g., a USB port) of the system 300.

Stimulator(s) 307 may include high voltage generators, amplifiers, switches, controlled-current and/or controlled-voltage sources, clocks, current and/or voltage-limiting elements, or other elements to controllably generate currents, voltages or other energies that can be delivered to a user's lip(s) via the first electrodes 303. In some examples, the stimulator 307 can be configured to generate a single controlled current/voltage at a time, and to operate an array of switches to deliver that single controlled current/voltage to a specified stimulation electrode and return electrode of the first electrodes 303, with different levels of stimulation provided via multiple different stimulation electrodes by operating the array of switches in a time-division multiplexed manner. Additionally or alternatively, the stimulator(s) 307 could include multiple stimulator systems capable of generating respective controlled currents/voltages at a time. For example, an independent stimulator system could be provided for each stimulating electrode of the system 300.

### III. Example Methods

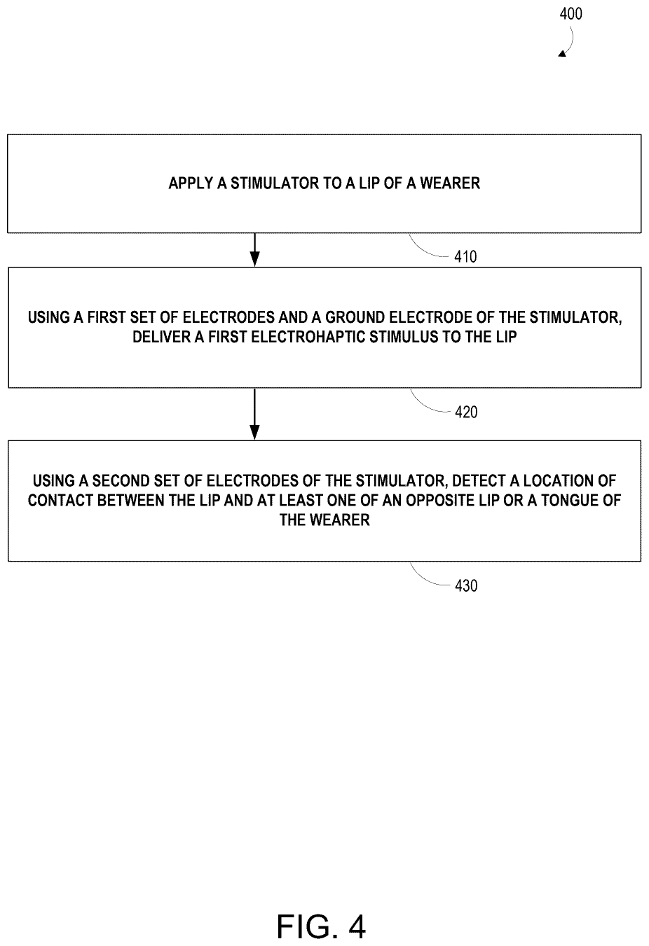

FIG. 4 depicts an example method 400. The method 400 includes applying a stimulator to a lip of a wearer (410). The method 400 additionally includes using a first set of electrodes and a ground electrode of the stimulator, delivering a first electrohaptic stimulus to the lip (420). The method 400 yet further includes using a second set of electrodes of the stimulator, detecting a location of contact between the lip and at least one of an opposite lip or a tongue of the wearer (430). The method 400 could include additional steps or features.

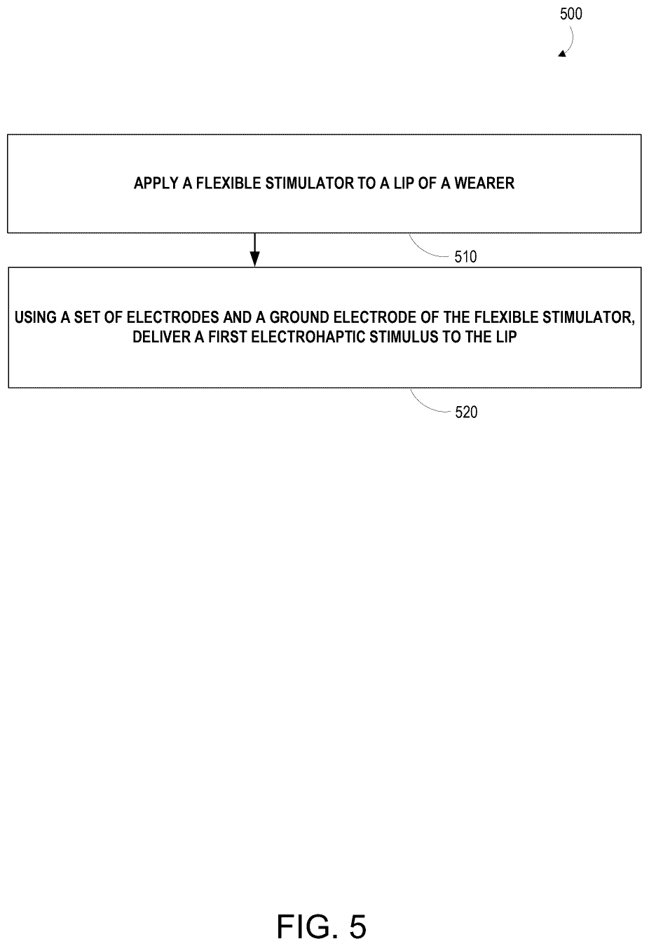

FIG. 5 depicts an example method 500. The method 500 includes applying a flexible stimulator to a lip of a wearer (510). The method 500 additionally includes using a set of electrodes and a ground electrode of the flexible stimulator, delivering a first electrohaptic stimulus to the lip (520). The method 500 could include additional steps or features.

It should be understood that arrangements described herein are for purposes of example only. As such, those skilled in the art will appreciate that other arrangements and other elements (e.g. machines, interfaces, operations, orders, and groupings of operations, etc.) can be used instead of or in addition to the illustrated elements or arrangements.

### IV. Experimental Results

As an example of the embodiments described here, an experimental system called “LipIO” was constructed and experimentally evaluated. LipIO allows the lips to be used simultaneously as an input and output surface. LipIO includes two overlapping flexible electrode arrays: an outward-facing array for capacitive touch sensing and a lip-facing array for electrotactile (which may alternatively be referred to as electrohaptic) stimulation. While wearing LipIO, users perceive the interface's state via lip stimulation and respond by touching their lip with their tongue or opposing lip. By providing sensing and stimulation electrodes that correspond to each other and overlap in a pairwise fashion, LipIO can be operated to provide co-located tactile feedback to allow users to know where along the LipIO device on the lip they are touching—this facilitates eyes- and hands-free interaction via the LipIO device. Three studies verified that participants were able to perceive electrotactile output on their lips and subsequently to touch the stimulated target location with their tongue with an average accuracy of 93%, using the LipIO with five I/O electrodes and co-located feedback stimulation. The utility of LipIO in four exemplary applications was investigated, showing that the embodiments described herein can facilitate new types of eyes- and hands-free micro-interactions.

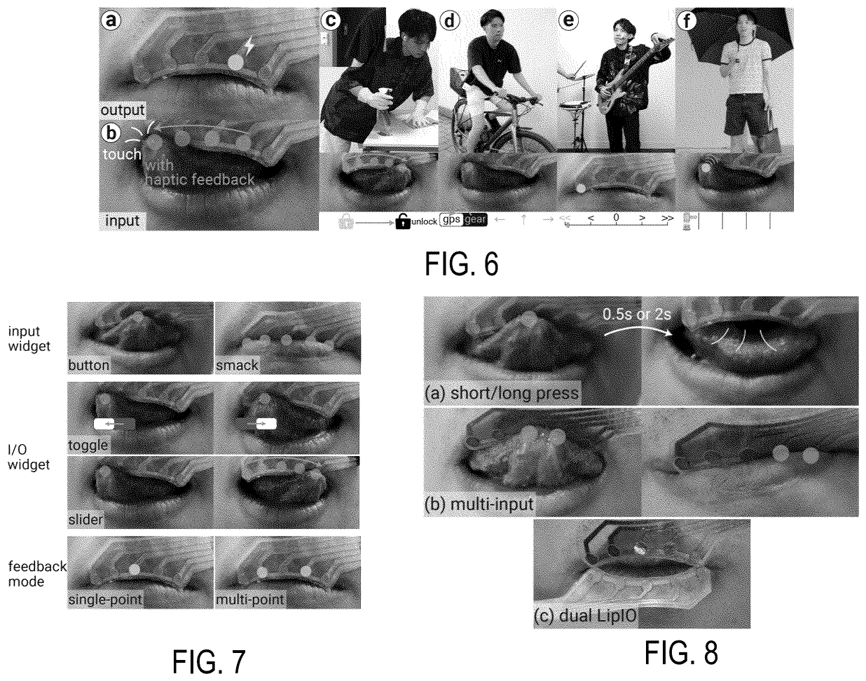

FIG. 6: LipIO enables the user's lips to be used simultaneously as an input and output surface. It supports eyes- and hands-free interactions via (a) electrotactile stimulation as output and (b) capacitive touch as input. A range of practical applications were experimentally demonstrated, including (c, d) I/O for controlling appliances (e.g., a smart door or an e-bike's GPS and lights), (e) outputting the state of an interface (e.g., feeling the output of a guitar tuner rather than seeing it), and (f) rendering a simple game entirely on the lips (e.g., whack-a-mole).

The eyes and hands are the primary way many users interact with their environment, including electronic devices such as smartphones or other computers. As such, it is common for users to find themselves in many everyday situations where their eyes and/or hands are occupied and not available to interact with such interfaces.

As an alternative to the more traditional modalities that can often be overloaded in everyday situations (as is the case of a user's eyes, hands, or ears), intra-oral interfaces have been investigated. These devices reside inside the user's mouth, typically atop the user's tongue or roof of the mouth. These have been used for tongue/teeth input but also for output, e.g., electrical stimulation on the tongue or electrical stimulation on the upper palate. However, realizing both input and output inside the oral cavity has proven elusive, and only a few assistive devices have been demonstrated. Additionally, such devices are often incompatible with eating/speaking (e.g., due to including thick retainers with cables coming out of the mouth or other elements that impede speech/feeding).

LipIO avoids encumbering the user's eyes/ears/hands and avoids encumbering the user's ability to speak or eat by using the user's lips as both an input and output surface for eyes- and hands-free interactions. LipIO closes the tactile input and output loop for lip-based interactions by co-locating the input and output directly at the user's lips. LipIO takes advantage of (1) the sensitivity of the lips—the lips are as sensitive as the fingertips—which is leveraged to repurpose the lips as an output surface via electrotactile stimulation; and (2) the dexterity of the tongue—the tongue (and also the lips) are exceptionally dexterous body parts, in many ways comparable to fingers. LipIO (FIG. 6) is an I/O device comprised of two flexible sandwiched electrode arrays: an outwards-facing array for capacitive touch and an inward-, lip-facing array for stimulation. Thus, while wearing the LipIO device, users can feel the state of a user interface by means of electrotactile stimulation of their lips (FIG. 6a), and they can respond by touching the perceived locations using their tongue (FIG. 6b) or the lower, opposite lip for coarser input. Whenever the user touches their lips to input, they can be provided with co-located tactile feedback at the location that was touched. This configuration facilitates eyes-and hands-free interaction.

Three user studies were conducted to quantify the use of a LipIO-like system for input and output via the tongue and the lips. Participants were able to perceive output on their lips and subsequently touch the same location with their tongue with an average accuracy of 93%, while wearing a LipIO device with five I/O electrodes and co-located tactile feedback.

Four applications were implemented to demonstrate different uses of LipIO in everyday contexts: outputting the state of an interface via the lips (e.g., a guitar tuner application), controlling appliances via input & output on the lips (e.g., an application that allows a user to control a smart door, or an e-bike application that allows the user to switch between controlling the lights or GPS), and simple games played entirely via input and output through the user's lips (e.g., a whack-a-mole game).

Additional interactive uses for LipIO were also demonstrated to investigate the use of LipIO as device for accessibility research, as a haptic interface, or as a supplementary modality.

LipIO builds on the high tactile acuity of the lips as the output surface and the high dexterity of the tongue as the input.

FIG. 1: Interaction paradigm in LipIO. The lips are used as input and output surface to control a target application.

Human lips are as or more sensitive than fingers in the grating orientation discrimination test. Lips also display a high tactile acuity in electrotactile stimulation.

The tongue has a high tactile sensitivity rivaling that of the fingers. The tongue can form different shapes using ten different muscles. The tongue also displays a relatively fast reaction time of around 600 ms. These capabilities facilitate the essential roles played by the tongue in speaking and eating. The area of the cerebral cortex that corresponds to the tongue is one of the largest in the primary sensory and motor cortical areas.

FIG. 1 depicts aspects of the operation and design of LipIO: (1) a user controls an external interactive device by using their lips as an input surface (via touch input on lips, via tongue or opposite lip); and, conversely, (2) the interactive device responds and informs the user by using their lips as an output surface (via electro-tactile stimulation of the lips).

Thus, LipIO can be used to implement a symmetric I/O interaction with an interactive device, in which users input and output using a similar UI vocabulary on the same surface. This application of symmetric I/O to the lips facilitates the creation of new types of interfaces that may be especially useful in eyes- and hands-busy situations. Additionally or alternatively, a lip-based interface as described herein can be used as a supplement to existing modalities (e.g., LipIO+speech I/O, LipIO+gestures, etc.), as an accessibility I/O interface, and so forth.

To demonstrate user interactions via the lips, four interactive widgets were implemented (depicted in FIG. 7), which were inspired by those found in conventional GUIs. When the user provides inputs via any of these widgets, they may concurrently receive feedback from LipIO in the form of electrotactile stimulation at the touched location, allowing users to feel where on the interface they are touching. The four interactive lip widgets were: (1) momentary push button as an input widget, created from a single I/O electrode pair, allowing users to trigger actions when a sensing electrode is touched; (2) smack as an input widget, created from all I/O electrode pairs, allowing users to activate/deactivate/switch LipIO applications by smacking their lips together (similar to saying “pa” in English); (3) toggle as an I/O widget, created from two adjacent I/O electrode pairs, allowing switching between two values (by sliding to the left or right electrode) or reading its state (by touching it, the user can feel which one is active, left or right); and (4) slider as an I/O widget, created from at least three adjacent I/O electrode pairs, allowing the user to input linear values (by sliding the multiple electrodes in to left or right and stopping at the desired level) or to read a state (by feeling which electrode is the last in the stimulation sequence).

Interactions with LipIO can benefit from two distinct electro-tactile stimulation modes: (1) single-point feedback, in which users feel one electrode at a time (e.g., button press, toggle slide, slider swipe)—as shown in the various applications herein, even just a single point of stimulation can yield expressive feedback as the temporal profile can be controlled, e.g., rendering spatiotemporal patterns; and (2) multi-point feedback, in which users feel two or more electrodes being stimulated concurrently (e.g., rendering simple animations on the lips, vibrating all points to confirm a lip-smack gesture, etc.). This was implemented technically by time-based multiplexing, switching rapidly between stimulation electrodes.

FIG. 8: Additional interactive techniques can be implemented using LipIO: (a) short vs. long press; (b) allowing touching adjacent targets by touching the upper lip with a wider region of the tongue or with the lower lip; and (c) using both lips as I/O surfaces. LipIO-based applications can also leverage two interactive techniques typically found in touch-based interfaces (depicted in FIG. 8): (a) differentiating between short (<0.5 s) and long (>2 s) presses; and (b) allowing users to touch adjacent targets by touching the upper lip with a wider region of the tongue or with the lower lip. Finally, while several example implementations depicted herein are limited to the use of a single LipIO device disposed on the upper lip (this being is the most ergonomic touch location for the tongue), specialized applications can use both lips as I/O surfaces by wearing two of the devices as described herein, increasing the number of I/O channels, e.g., to 10.

A system as described herein provides a number of benefits: (1) it provides eyes- and hands-free interface; (2) it is flexible and fits the curve of the lip; (3) it features co-located tactile feedback, leading to increased usable I/O accuracy (93%); and (4) it does not drastically interfere with everyday tasks such as speaking and eating.

Note that, while the example devices depicted herein are visible against the lip, it is possible to use transparent electrotactile actuators and sensors, and other reduced-visibility elements, to reduce the conspicuousness of a LipIO or other system as described herein.

Four applications were implemented to evaluate the efficacy of the embodiments described herein: (1) an interface to unlock a smart door; (2) a guitar tuner; (3) an e-bike interface; and (4) a lip-based “whack-a-mole” game. These four applications represent examples of applying LipIO to everyday situations in which users might find their eyes and hands occupied with primary tasks but simultaneously desire to control an interface.

1) Unlocking a Smart Door While Eyes- & Hands-Busy

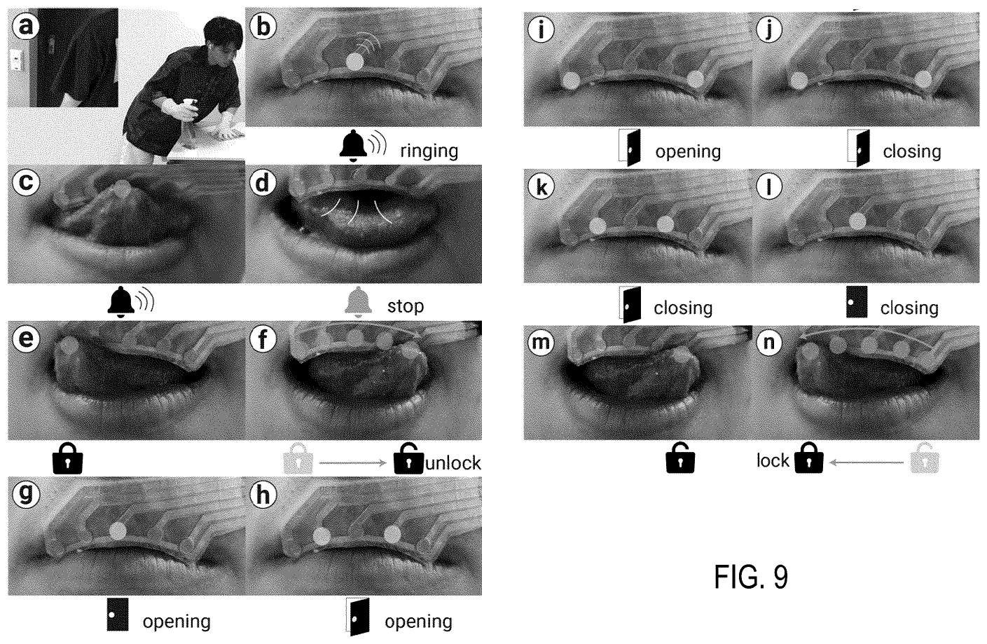

In this example, LipIO's tactile stimulation allows simple animations to be rendered on the lip that create useful interface metaphors. FIG. 9 shows a user who is listening to music while cleaning—their hands, ears, and eyes are occupied with these primary tasks. FIG. 9 depicts the whole interaction: (a) a visitor rings the door; (b) LipIO renders the “ringing” via its tactile feedback (vibration at 3 Hz) at the center electrode—this vibration is a typical UI metaphor for ringing; (c) in response, the user touches the ringing electrode to (d) stop the bell ringing. Now, to open the door, (e) the user swipes from left to right—a typical UI metaphor for sliding a door open. Now, LipIO renders the state of the door, so the user feels as their friend walks in: (g→i) the stimulation fans out from the center electrodes every 700 ms—a metaphor for opening; and then, as their friend closes the door, (j→l) the opposite animation is rendered to the electrodes. Finally, (m) locks the door by repeating the swipe gesture in the opposite direction of their initial unlock.

2) A Hands-Free & Eyes-Free Guitar Tuner

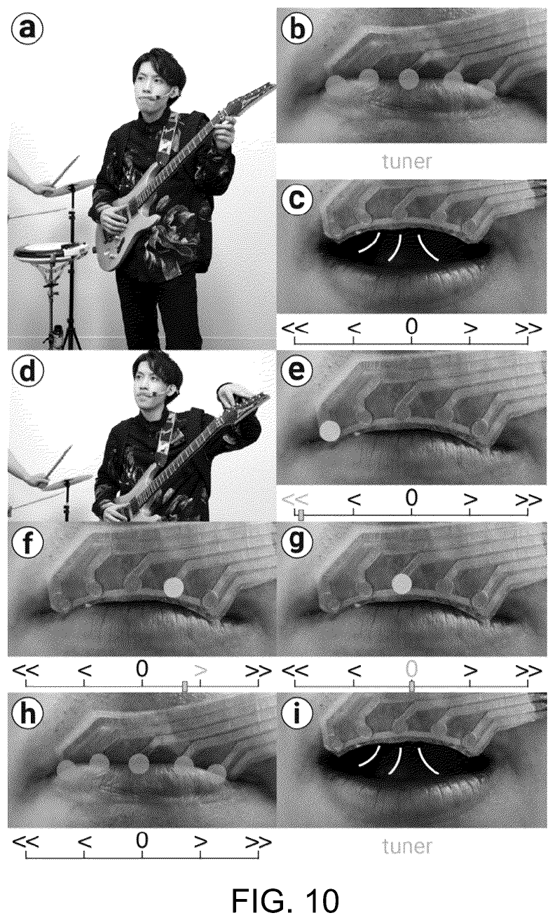

The second application demonstrates how LipIO can apply its electro-tactile output of the user's lips to enable new types of hands- & eyes-free interactions. In this case, the user is tuning their guitar while freely moving on stage and keeping eye contact with the audience—something not possible with existing tuners which require eye contact. The LipIO tuner simulates the visual display of existing tuners, which depict visually which direction the string's frequency deviates from the expected note (up, down, or tuned)—the LipIO implementation follows this layout to leverage familiarity. FIG. 10 depicts this interaction: (a) the user plays while looking at the audience; the user invokes the tuner using the lip-smack: (b) to do this, they join their lips together and feel co-located tactile feedback to confirm this gesture has been detected. Then, they (c) smack the lips (as if saying the sound “pa”); (d) now, as the user tunes the string with their hands, (e) LipIO stimulates the leftmost point in response, indicating this string is out of tune, too low; then, (f) LipIO responds by indicating that the string is now slightly overpitched, which the user corrects by lowering; (g) finally, LipIO indicates that the string is tuned; (h) the user repeats the lip-smack gesture to (i) dismiss LipIO.

3) Multi-Page Interface: Switching Between Two Applications of an E-Bike

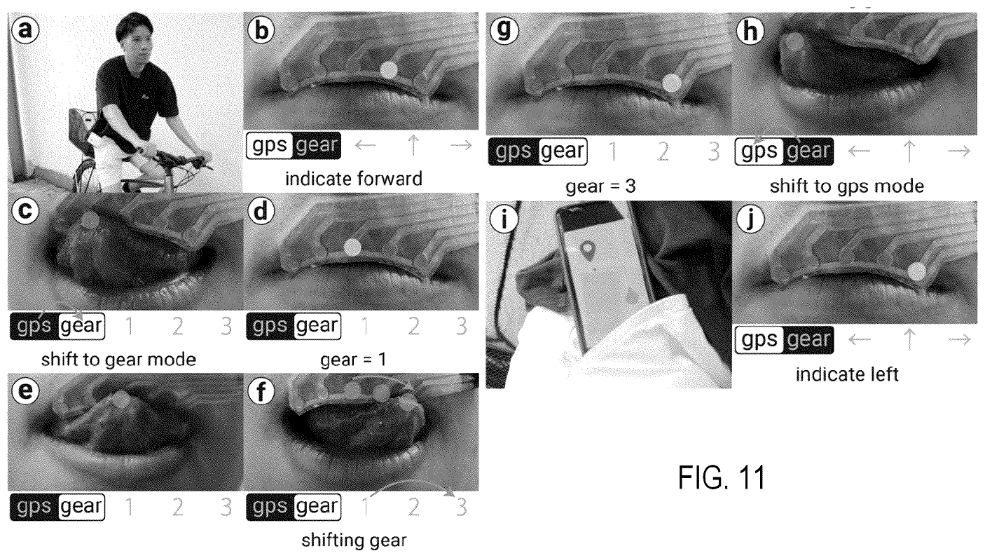

While the experimental LipIO device included electrode arrays (input and output) of only five channels each, ensuring an I/O accuracy of over 90%, some micro-interactions might be improved by the use of more than five electrodes. One way to implement such an interface is by adopting a multi-page layout. To demonstrate this, in the third application, a toggle interface switches between two UI pages, each featuring different UI elements of the user's e-bike: a GPS navigation page and a gear settings page. FIG. 11 depicts the interaction: (a) the user is biking while wearing LipIO; (b) the selected UI page is the GPS, informing the user to keep moving forward, which they feel by means of the stimulating electrode (the center of the three navigation electrodes). Then, (c) the user switches to the gear application by touching and “flipping” the toggle with their tongue; (d) now, in the gears page, they feel that their e-bike is in the lowest setting (least motor assistance); the user adjusts the gear by (e) touching the current gear and (f) sliding the tongue to the next gear; until, (g) the e-bike is now the highest gear (maximum motor assistance). Finally, (h) they switch to the GPS page, and (j) they feel the GPS indicating a left turn.

4) Lip-Based Game Interface: Playing “Whack-a-Mole” Game

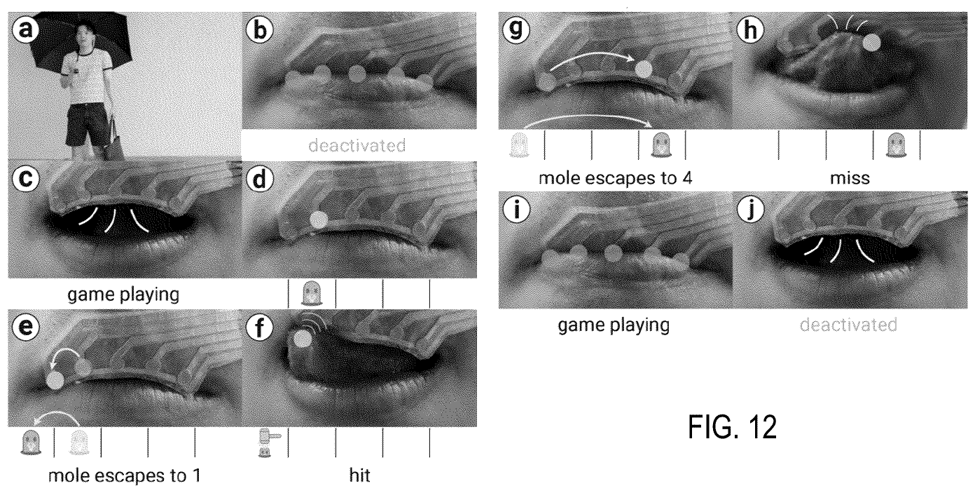

Our fourth application (shown in FIG. 12) utilizes the lips as I/O surfaces to render a gaming experience. Most game UIs require users to focus their eyes, ears, and hands on the game screen. Instead, in this application our user is playing a “whack-a-mole” game while searching for their friend and holding luggage; thus, their eyes and hands are busy).

The experimental LipIO system included three main components: (1) a flexible sensing electrode array, (2) a flexible electrotactile actuation electrode array, and (3) electronic circuitry. Note that the particulars of implementation of the LipIO system intended only as an illustrative example of the embodiments depicted herein; the subject matter of the present disclosure extends beyond these particulars to alternatives as will be evident to one of skill in the art.

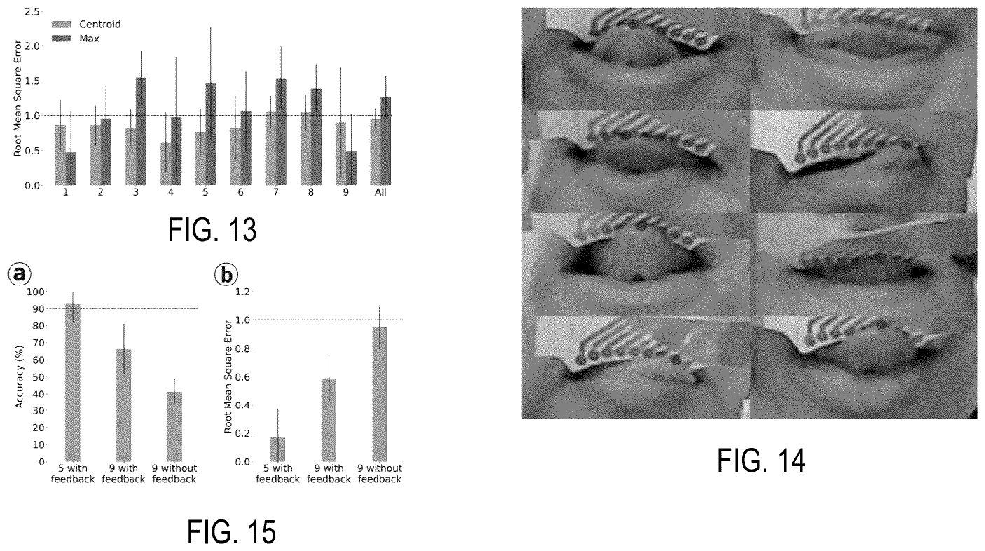

Individual users can be provided with their own flexible electrode arrays for comfort and hygiene. To speed up the fabrication time of and reduce the cost of producing such arrays, conductive inkjet printing can be employed. A commodity inkjet printer (EPSON PX-S160T) was used to print a silver nanoparticle-based ink (Mitsubishi NBSIJ-MU01) on a white opaque coated paper (Mitsubishi NB-RC-3GR120). Alternatively, to produce more resilient, longer-lasting partially-transparent arrays (which are depicted in the various Figures of the drawings other that FIG. 14), copper tape (LOVIMAG) was cut using a craft cutter (Cricut Explore Air 2). This approach lasted longer than the screen-printing approach, and was more robust to bending. Each electrode array was taped to a flexible cable via z-axis conductive tape (3M) and connected to the electrical circuit. An end-to-end resistance of ˜0.5Ω was measured from the electrodes to each of their path ends.

Two electrode arrays (one input, and one output) were stacked together, as depicted

in FIG. 2. To prevent the output layer and the lip surface from affecting the capacitive sensors, an isolating flexible substrate layer of plastic sheet (135 μm) was disposed between the layer of input electrodes and the layer of output electrodes. A temporary tattoo paper (Silhouette) was placed over the input electrodes to prevent the tongue from directly touching the input electrodes. A laser-cut adhesive sheet (Silhouette, 140 μm) was then disposed atop that layer to insulate the non-electrode parts from the tongue and thus reduce incorrect touch recognition. FIG. 2(b) shows the path design for the input and output electrode arrays. FIG. 2(c) shows the whole device. The resulting stacked and co-located array was 360 μm thick and flexible to match the curve of the user's mouth, as depicted in FIG. 2(d).

To attach the flexible stimulator array (and the electrodes thereof) to the user's lip, another laser-cut adhesive sheet was disposed on the layer of output electrodes. After using the device, the adhesive strength can be restored by re-applying another adhesive sheet of the same shape.

The particular dimensions and other aspects of a device as described herein can be adjusted to obtain different benefits and/or to adapt these embodiments to a particular user or application. For example, decreasing the electrode radius beyond what is shown can result in decreased conductivity and subsequently decreased sensor/actuator response but can also allow for an increased number of electrode channels to be disposed in the device. Thinner trace widths can also be used, resulting in decreased conductivity but also a reduced device size and/or increased channel count. As a practical example, 4 mm electrodes can be used—this allows for at least ˜9 electrodes in a single LipIO device.

To effect capacitive sensing, an NXP MPR121 (on-chip capacitive touch controller with 12 independent channels) was used, which exhibits high dynamic range (measuring changes in electrode capacitance ranging from 1 pF to 2000 pF). This allowed the experimental system to detect different types of touches, such as lip-to-lip or tongue-to-lip. The MPR121 also provides an onboard hysteresis filter and dynamic baseline calibration, reducing false positives caused by residual saliva left after tongue touches.

For electro-tactile stimulation, the medical-compliant Hasomed RehaStim was used; however, other stimulators may be used, such as smaller devices like the Biosync wearable stimulator or a custom-designed stimulator circuit or apparatus. To control which electrode outputs the stimulation, an array of Sharp PC817X×NSZ1B photo-relays was used, arranged in a 1:N multiplexer configuration. These photo-relays are rated for up to 80V at 50 mA of current, which is 10× the typical current values used for tactile stimulation. In the studies depicted herein, only 0.5 mA-10 mA was used, set according to the participants' comfort. The channel multiplexer affords a response time of 4 s, enabling switching between channels or even “simultaneously” stimulating multiple channels via time-multiplexing.

Three user studies were conducted. (1) The first study investigated which of the lips is easier to touch with the tongue; in about 90.0% of the trials, participants found the upper lip was easier to touch with their tongue. Accordingly, the upper lip was used in subsequent studies. (2) The second study provided participants with electrotactile stimuli in their lips and the participants then attempted to touch the perceived position of the stimuli with their tongue. This task was performed without tactile feedback and eyes-free, requiring participants to rely only on tongue proprioception and lip tactile feedback during tongue touch. This study illustrated the basics of tongue-to-lip interactive touches. The raw data of participants' touches were used to assess which common touch sensing approach (maximum-value or centroid) was most suited for tongue-to-lip touches. A centroid-based approach was better at allowing participants to estimate where the tongue was touching on the lip. This centroid-sensing method was then implemented in the experimental system to allow users to feel co-located output at positions being touched by their lips/tongues. (3) The third study investigated the varying numbers of electrodes and the parameters for providing co-located feedback. Adding co-located feedback improved the participants' accuracy from an initial average of 41.3% (without co-located feedback) to an accuracy of around 66.3%. Reducing the number of I/O electrodes from 9 to 5 further improved the participants' average accuracy from 66.3% to an accuracy of 93.1%, which was deemed usable for an interface to implement the various experimental applications.

### Study (1)

A preliminary study was performed to assess which of the lips is easier to reach with the tongue—this can be used to determine which lip is more suitable for tongue touches, thereby informing subsequent design decisions, implementations, studies, and applications.

8 participants were recruited without motor impairments on the lips or tongue (M=24.6 years old, SD=2.6; three identified as female and five as male). Five equally spaced 2 mm×2 mm tape squares were applied on the participants' upper and lower lips (spacing was relative to each participant's lip length). Participants were instructed to touch a pair of positions (upper vs. lower) with their tongue (no mirrors, task was performed eyes-free) and chose which, upper vs. lower lip, was easiest to touch. Each participant conducted 15 trials (5 pairs×3 repetitions) in a randomized order for a total of trials across all participants.

Most trials showed that the upper lip was easier to touch (average of 90.0%; SD=9.4). This is in line with the ergonomics of the tongue since the protrusion movement of the tongue outside of the oral cavity is minimal if the tongue touches the upper lip. Conversely, the protrusion is longer and more demanding for touching the lower lip. None of the participants voiced discomfort or inability to touch the lower lip; alternative implementations of LipIO-type devices can be used that include electrodes on both upper and lower lip.

### Study (2)

This experiment gathered participants' tongue touches on different lip targets to quantify how the user touches their lips with their tongue in an eyes-free manner and to devise parameters for high accuracy tongue touch. This task was performed with no-tactile feedback and eyes-free, requiring participants to rely on tongue proprioception and lip tactile feedback during tongue touch. Participants wore the LipIO device while sitting down, with their head supported by a chinrest. A keypad was placed near the participants' dominant hand to confirm their inputs. 9 electrode arrays were used for this study, which was the maximum number of electrodes that could be robustly fabricated at the time; with 4 mm circular electrodes located 4.7 mm apart. In a single trial, participants felt electrotactile stimulation in one of the nine locations. Then, they were instructed to, eyes-free (no mirrors or other means for visually observing their lips/tongue), touch with their tongue the point at which they perceived the stimulus (in any way they preferred) and press a button to confirm the location. The button was pressed, the apparatus recorded a photograph of their face (with their tongue sticking out at their chosen touch location) and the capacitive sensor values for all the sensing electrodes obtained from the touch sensor controller was also recorded. Participants performed 36 trials (9 points×4 repetitions). Locations were presented in randomized order. This totaled 288 trials across all participants (each trial composed of 9 data points from the touch sensors and one image of the participant's tongue touching the sensor array, for a total of 2592 touch values and 288 images).

Prior to the start of the trials, the intensity of the electro-tactile stimulation was calibrated for all nine points to ensure pain-free operation. The intensity ranged from a minimum of 0.5 mA up to the value that the participant deemed clearly noticeable and comfortable. Sub-mA adjustments were also conducted, when necessary, by adjusting the stimulation pulse-width from 50 s up to 300 s (in steps of 50 s). After calibration, an average intensity of 2.3 mA (SD=0.9 mA; median=2 mA) with a pulse-width of 223 s (SD=73 s; median=200 s) was used.

A 1080 p camera was used to gather images of participants' lips. The camera feed was corrected for distortions via a fisheye lens calibration software using a printed checkerboard pattern.

Eight new participants (M=22.9 years old, SD=2.2; five identified as male, three as female) were recruited.

FIG. 13 depicts the results of this study, showing the root mean square error (RMSE) for two possible touch estimators: in pink, a maximum-value estimator (i.e., an estimator that outputs as the touched location the highest value sensor reported from all the electrodes) and in blue, a centroid estimator (i.e., an estimator that outputs as the touched location the centroid of all the sensor values).

The error was lower for the centroid estimator (M=0.95; SD=0.15) when compared to the maximum estimator (M=1.27; SD=0.29). These error values were calculated using the electrode index (1-9). For example, a value of “1.0” means that the stimulating electrode and the touched electrode were off by one electrode. These results indicate that a centroid-based approach performed better than maximum-value for tongue-to-lip touches.

FIG. 14 depicts examples (one per participant) in which the correct target (annotated in blue) was touched. In these examples, the tip of the tongue (annotated in pink) was the closest anatomical feature to the target electrode. However, while the tip is touching the target electrode, the surrounding tongue areas are also touching adjacent electrodes-this happens because the tongue was wider than the electrodes. This may be a partial cause for the centroid estimator outperforming the maximum value estimator: since the tongue lands fairly symmetrically during lip touches, the tip tends to be found closest to the center of the raw touch data, which the centroid estimates better.

The RMSE had an average of 0.95 (SD=0.15), suggesting that participants tended to be off by about one target in these trials. In a practical implementation of a lip-based HCI as described herein, this error can be reduced by providing co-located tactile feedback that provides stimulation at the detected location at which the user's tongue touches the interface. In these trials, which lacked such feedback (and which were eyes-free and nearly tactile-free), an average low accuracy of 41.3% (SD=7.7) was measured.

### Study (3)

Using the centroid-based estimator, the performance of LipIO as an HCI in real-time was evaluated by providing co-located tactile feedback anytime the lips were touched. This allowed for the quantification of (1) the impact on touch location accuracy provided by the co-located tactile feedback, and (2) the impact on accuracy provided by reducing the number of touch locations from nine to five. The same apparatus as in the prior study was used, that participants received co-located tactile feedback. This feedback was determined in real-time using the centroid location estimator.

We followed the same trial design of Study 2 was used (i.e., participants felt a tactile stimulation in one of the possible locations and were asked to touch the point they felt with their tongue, confirming with a button press). The 9-point electrode array from Study 2 and an additional 5-point electrode array were used to quantify the effect of reducing the number of possible touch points.

The same procedure & calibration from Study 2 was used. After calibration, an average intensity of 3.1 mA (SD=1.8 mA; median=2 mA) with a pulse-width of 145 s (SD=92 s; median=100 s) was observed, similar to Study 2, with the median intensity at the same level and the median pulse-width 100 s slightly lower—suggesting that individual calibrations were consistent. To directly compare the improvement over the baseline recorded in the previous study, the same participants from Study 2 were used.

FIG. 15 depicts the findings of this study. An accuracy of 66.3% (SD=14.9) was observed when using touch feedback with the 9-point array, shown in FIG. 15(a). Compared to the baseline of the previous study (41.3%), in which participants did not receive any tactile feedback, this suggests that touch feedback increased performance by 25.0%. As depicted in FIG. 15(b), the RMSE also decreased from 0.95 (SD=0.15) to 0.59 (SD=0.17); suggesting that participants often are one electrode or more away from the target.

For the 5-electrode array, the RMSE decreased further from 0.59 to 0.17 (SD=0.20). This reduction in electrode count further increased the performance by 26.8%, resulting in a final average accuracy of 93.1% (SD=11.2).

These results show an accuracy of around 93% for tongue-to-lip touches using LipIO. Adding the co-located tactile feedback was important, as it increased the accuracy by ˜25%. Participants had no prior training with the LipIO, excepting a few calibration trials to adjust the intensity of the electrotactile sensations—in practice, a user of such a system would likely perform even better with additional experience, and indeed could benefit from additional electrodes and/or closer electrode spacing.

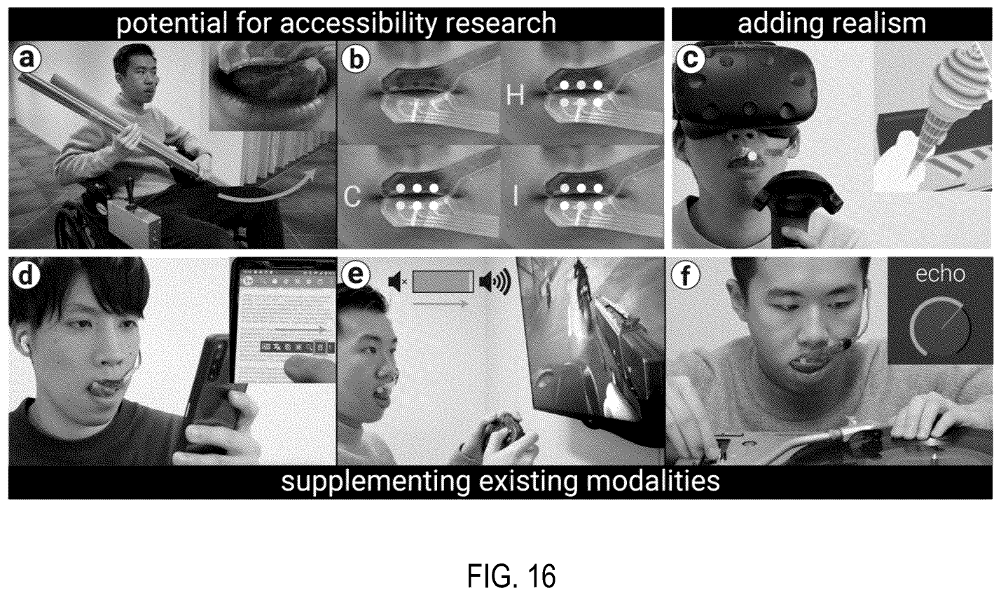

FIG. 16 depicts additional practical applications of the embodiments disclosed herein. These embodiments highlight three use cases: (1) LipIO as a potential device for accessibility research; (2) LipIO for enhancing realism; and (3) LipIO as a supplementary modality.

LipIO can be applied to the research in accessibility. In this domain, LipIO provides as least two advantages: (1) unlike existing intra-oral interfaces, which typically require attaching magnetic trackers to the tip of the tongue (typically via adhesives or piercings), LipIO is applied using skin-safe adhesives that are simpler to attach/remove; and (2) LipIO presents users with an input surface that can also render collocated output. FIG. 16(a) shows a user controlling their motorized wheelchair via LipIO. Tactile encoding of information is also possible. FIG. 16(b) shows how LipIO can be extended, for instance, to render braille to lips, allowing users to read braille via their lips using LipIO.

Another use for a LipIO-like system is as a haptic interface to increase the realism of virtual interactions. For instance, FIG. 16(c) shows a use of the LipIO to render a more realistic sense of tasting virtual ice cream. While existing methods to deliver tongue stimulation require cumbersome electrodes directly clipped to the tongue, LipIO can leverage its vantage point to stimulate the tongue by momentarily switching the sensor, via a multiplexer, to act as another electrotactile actuator whenever the user licks the sensor.

LipIO-like systems can also work as a supplementary HCI modality that enriches an existing interaction. For instance, FIG. 16(d) shows LipIO used as a supplementary modality for interacting with a touchscreen; in this interaction, sliding the tongue controls the contextual pop-up menu of options to the user's currently touched location. FIG. 16(e) shows LipIO as a supplementary modality to control the volume of a videogame experience without the need to invoke sub-menus or use a remote controller. FIG. 16(f) shows LipIO as an additional input for a DJ application—a situation in which users often desire additional inputs since they are typically undertaking several simultaneous tasks. In this example, the DJ uses LipIO to control the amount of “echo” while simultaneously adjusting the tempo of a secondary song they are about to mix in.

### V. Conclusion

It should be understood that arrangements described herein are for purposes of example only. As such, those skilled in the art will appreciate that other arrangements and other elements (e.g., machines, interfaces, operations, orders, and groupings of operations, etc.) can be used instead, and some elements may be omitted altogether according to the desired results. Further, many of the elements that are described are functional entities that may be implemented as discrete or distributed components or in conjunction with other components, in any suitable combination and location, or other structural elements described as independent structures may be combined.

While various aspects and implementations have been disclosed herein, other aspects and implementations will be apparent to those skilled in the art. The various aspects and implementations disclosed herein are for purposes of illustration and are not intended to be limiting, with the true scope being indicated by the following claims, along with the full scope of equivalents to which such claims are entitled. It is also to be understood that the terminology used herein is for the purpose of describing particular implementations only, and is not intended to be limiting.

We claim:

1. A system comprising a stimulator, wherein the stimulator comprises:
a flexible substrate having a first surface and a second surface opposite the first surface;
a first set of electrodes disposed on the first surface of the flexible substrate;
a ground electrode disposed on the first surface of the flexible substrate; and
a second set of electrodes disposed on the second surface of the flexible substrate,
wherein the stimulator is mountable to a lip, wherein the first set of electrodes and ground electrode are operable to provide electrohaptic stimulation to the lip when the stimulator is mounted thereto, and wherein the second set of electrodes are operable to detect a location of contact between the lip and at least one of an opposite lip or a tongue when the stimulator is mounted to the lip.

2. The system of claim 1, further comprising:
a controller comprising one or more processors and configured to perform controller operations including:
applying electrohaptic stimulation through a first electrode of the first set of electrodes, using the ground electrode as a return electrode; and
using the second set of electrodes, detecting a location of contact between the lip and at least one of the opposite lip or the tongue.

3. The system of claim 2, wherein using the second set of electrodes to detect a location of contact between the lip and at least one of the opposite lip or the tongue comprises detecting the capacitance of two or more electrodes of the second set of electrodes.

4. The system of claim 2, wherein the controller operations further comprise:
during a first period of time, using the second set of electrodes to detect a location of contact between the lip and at least one of the opposite lip or the tongue;
based on the detected location of contact during the first period of time, determining that a user performed a first gesture during the first period of time;
responsive to determining that the user performed the first gesture during the first period of time, transmitting an indication of the first gesture to a remote system;
during a second period of time, using the second set of electrodes to detect a location of contact between the lip and at least one of the opposite lip or the tongue;
based on the detected location of contact during the second period of time, determining that the user performed a second gesture during the second period of time, wherein the second gesture differs from the first gesture; and
responsive to determining that the user performed the second gesture during the second period of time, transmitting an indication of the second gesture to the remote system.

5. The system of claim 4, wherein determining that the user performed the first gesture during the first period of time comprises at least one of: (i) determining that the user moved the location of contact from one end of the second set of electrodes to an opposite end of the second set of electrodes, or (ii) determining that the user contacted a specified set of one or more electrodes of the second set of electrodes without contacting any other electrodes of the second set of electrodes.

6. The system of claim 2, wherein the controller operations further comprise:
receiving an indication of a first output from a remote system;
responsive to receiving the indication of the first output, providing, via the first set of electrodes, a first pattern of electrohaptic stimulation that corresponds to the first output;
receiving, from the remote system, an indication of a second output;
responsive to receiving the indication of the second output, providing, via the first set of electrodes, a second pattern of electrohaptic stimulation that corresponds to the second output, wherein the first pattern differs from the second pattern.

7. The system of claim 2, wherein applying electrohaptic stimulation through the first electrode comprises applying less than 5 milliamps of current through the first electrode.

8. The system of claim 2, wherein the controller operations further comprise:
applying electrohaptic stimulation through a second electrode of the first set of electrodes, using the ground electrode as a return electrode, wherein the first electrode differs from the second electrode.

9. The system of claim 1, wherein the second set of electrodes consists of four to nine electrodes.

10. The system of claim 1, further comprising an insulator layer disposed between the first set of electrodes and the ground and the lip when the stimulator is mounted to the lip, wherein the insulator layer has a plurality of holes formed there through such that each electrode of the first set of electrodes can provide electrohaptic stimulation to the lip when the stimulator is mounted to the lip.

11. The system of claim 1, further comprising an insulator layer that is disposed between the first set of electrodes and the ground and the lip when the stimulator is mounted to the lip and that is configured to adhere the stimulator to the lip.

12. The system of claim 1, wherein each electrode of the first set of electrodes is composed of a metal foil disposed on the flexible substrate, wherein a particular metal foil of which a respective electrode of the first set of electrodes is formed also includes a conductive trace.

13. The system of claim 1, wherein each electrode of the first set of electrodes at least partially overlaps with a respective electrode of the second set of electrodes.

14. A method, comprising:
applying a stimulator to a lip of a wearer;
using a first set of electrodes and a ground electrode of the stimulator, delivering a first electrohaptic stimulus to the lip; and
using a second set of electrodes of the stimulator, detecting a location of contact between the lip and at least one of an opposite lip or a tongue of the wearer.

15. The method of claim 14, further comprising:
using a first subset of the first set of electrodes to deliver a second electrohaptic stimulus to the lip; and
using a second subset of the first set of electrodes to deliver a third electrohaptic stimulus to the lip, wherein the first subset and the second subset do not contain any of the first set of electrodes in common.

16. The method of claim 14, further comprising:
during a first period of time, using the second set of electrodes to detect a location of contact between the lip and at least one of the opposite lip or the tongue;
based on the detected location of contact during the first period of time, determining that a user performed a first gesture during the first period of time;
during a second period of time, using the second set of electrodes to detect a location of contact between the lip and at least one of the opposite lip or the tongue; and
based on the detected location of contact during the second period of time, determining that the user performed a second gesture during the second period of time, wherein the second gesture differs from the first gesture.

17. The method of claim 16, wherein determining that the user performed the first gesture during the first period of time comprises determining that the user moved the location of contact from one end of the second set of electrodes to an opposite end of the second set of electrodes.

18. The method of claim 16, wherein determining that the user performed the first gesture during the first period of time comprises determining that the user contacted a specified set of one or more electrodes of the second set of electrodes without contacting any other electrodes of the second set of electrodes.

19. The method of claim 16, further comprising:
responsive to determining that a user performed a first gesture during the first period of time, using the first set of electrodes and the ground electrode to deliver a feedback electrohaptic stimulus to the lip, wherein the feedback electrohaptic stimulus corresponds to the first gesture.

20. A method, comprising:
applying a flexible stimulator to a lip of a wearer; and
using a set of electrodes and a ground electrode of the flexible stimulator, delivering a first electrohaptic stimulus to the lip.
